(19) United States
(12) Patent Application Publication
(10) Pub. No.: US 2006/0111739 A1
(43) Pub. Date: May 25, 2006

(54) Device for stabilising and/or positioning a medical tool in a body cavity

(76) Inventors: Urs Staufer, Neuchatel (CH); Raphael Imer, Marin (CH)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/525,858

(22) PCT Filed: Sep. 3, 2002
(86) PCT No.: PCT/IB02/03602

§ 371 Date: Sep. 2, 2005

(51) Int. Cl. A61M 29/00

(52) U.S. Cl. 606/192

(57) ABSTRACT

The present invention provides a device 20 for stabilising and/or positioning a medical tool inside a body cavity. Said device comprises an elongated tubular structure 1, at least one inflatable balloon 3,4 connected to at least one capillary tube 5 and inflatable by pressing a fluid and/or as into said tube and a means 7 for receiving a medical tool. Furthermore, the present invention relates to a method for stabilising and/or positioning a medical tool in a body cavity.

### TECHNICAL FIELD

The present invention relates to a device for stabilising and/or positioning a medical tool in a body cavity and a method for stabilising a medical tool in a body cavity.

### BACKGROUND ART

The current evolution in nanotechnology enables observations and measurements of sub-micron and nanosize objects by means of scanning probes. This scale range is attractive for analyzing diseased human tissues.

During a classical surgery session, the stabilization of the tool is entirely relaying on the support by the surgeon's hand. Various types of mechanisms outside the body have been designed to block the instrument in a desired position. These mechanisms reduce un-wanted movement of the tool's tip. However, the achieved stabilization is likely to be insufficient for nanoscale measurements.

Therefore, there is a need for a device allowing a sufficiently precise stabilisation and/or positioning of a medical tool in a body cavity so that nanoscale measurements on human tissues can be performed.

### DISCLOSURE OF THE INVENTION

Hence, it is a general object of the present invention to provide a medical device for stabilising and/or positioning a medical tool in a body cavity. Said device comprises an elongated tubular structure with an end for insertion in a body cavity, at least one inflatable balloon connected to at least one capillary tube and inflatable by pressing a liquid and/or gas through said capillary tube and a means for receiving a medical tool. Said at least one balloon is arranged at a distance from said medical tool.

The term “body cavity” as used herein encompasses not only any hollow space within a body or any of its organs but as well the space in joints or the lumen of blood and lymph vessels.

The term “medical tool” as used herein encompasses any kind of instrument which can be used for a surgical, diagnostic or therapeutical purpose. Examples of such tools are a nanotool and a AFM (Atomic Forced Microscope).

In a preferred embodiment of the present invention said elongated tubular structure further comprises at least one surface opening and said at least one inflatable balloon is located at said at least one surface opening, preferably at least two surface openings and at least two inflatable balloons located at said surface openings, more preferably at least four surface openings and at least four inflatable balloons located at said surface openings.

In a further preferred embodiment said surface openings are uniformly distributed along a circumference of said elongated tubular structure and the centres of all said surface openings are in the same distant from the end of the elongated tubular structure to be inserted in the body cavity.

In another preferred embodiment the means for receiving a medical tool is a recess having a polygonal profile.

In a further preferred embodiment said elongated tubular structure further comprises a means for forcing the balloons to expand outside said elongated tubular structure when the balloons are inflated. Preferably, said means for forcing the balloons to expand outside said elongated tubular structure is a ring.

In a further preferred embodiment said capillary tubes connected to said inflatable balloons are fixed to said means for forcing the inflated balloons outside said elongated tubular structure.

The device of the present invention is suitable for stabilising and/or positioning a medical tool, preferably a nanotool, more preferably a AFM (Atomic force microscope).

In a second aspect the present invention relates to a method for stabilising and/or positioning of a medical tool in a body cavity. Said method comprises the following steps:

Introducing a device comprising a medical tool and at least one inflatable balloon arranged in a distance from said medical tool in a body cavity, inflating said at least one balloon with a liquid and/or gas until said at least one inflated balloon contacts the inner surface of the body cavity and the medical tool is stabilised and/or positioned.

In a preferred embodiment of said method the inflatable balloons are inflated by a liquid, preferably a physiological liquid.

In another preferred embodiment of said method said device comprises at least two balloons, preferably at least four balloons.

In a further preferred embodiment of said method said body cavity is a joint, preferably a knee joint.

In a third aspect the present invention relates to a medical instrument comprising a device of the present invention for stabilising and/or positioning a medical tool and a medical tool, wherein said medical tool is a AFM.

The device for stabilisation and/or positioning of a medical tool of the present invention allows a precise stabilisation of medical tools such as e.g. a AFM tip which are very sensitive to vibrations and allows to perform measurements in a sub-micron and nanosize scale range in the human body e.g. on diseased human tissue. The data of said measurements of diseased human tissue can be used for diagnostic purposes and can help to select a suitable therapy method.

The careful regulation of the relative pressure in the balloons allows for a positioning the medical tool relative to the tissue to be examined. In fact, the inflation or the deflation of a balloon approaches or withdraws the medical tool from the sample. A combination of e.g. four balloons increases appreciably the precision of the technique. When the positioning is finished, the inflating system can be deactivated and so it does not interfere with the measurement.

### BRIEF DESCRIPTION OF THE DRAWINGS

The invention will be better understood and objects other than those set forth above will become apparent when consideration is given to the following detailed description thereof. Such description makes reference to the annexed drawings, wherein:

FIG. 1a shows a top plan view of the device inside of a knee. Inflated balloons 4 exert a small pressure on the kneecap 9 and on the tibia bone 10;

FIG. 1b shows an enlarged part of FIG. 1a;

FIG. 2 shows a perspective view of the device 20, showing the surface openings 2 in the elongated tubular structure 1 and flat balloons 3;

FIG. 3a shows a cross-sectional view of the device 20 with flat 3 balloons;

FIG. 3b shows a cross-sectional view of the device 20 with inflated balloons 4;

FIG. 4a shows a cross-sectional view of the device 20, using differential pressure in the inflatable balloons 4 for positioning the medical tool relative to the human tissue that is examined and

FIG. 4b shows a cross-sectional view of the device 20, using differential pressure in the inflatable balloons 4 for positioning the medical tool relative to the human tissue that is examined.

### MODES FOR CARRYING OUT THE INVENTION

FIG. 1a and b show an exemplary use of a device 20 for stabilising and/or positioning a medical tool in the knee 21 of a subject in order to perform in vivo AFM measurements.

As during a state of the art arthroscopy, several little incisions are made in the patient's knee, depending on the required tools. Through a first opening, the physiological liquid is introduced. Through a second one, an optical fibre based microscope, the arthroscope is introduced, which allows real-time and colour imaging of the inside of the knee. The device 20 with a nanotool or AFM can be introduced through a further incision in the same way as standard minimal invasive tools are employed.

After its insertion, the inventive device 20 can be guided to the right position with the help of the arthroscope. Then, the stabilization is performed by inflating the balloons 3,4 of the device 20. Balloons' expansion exert a small pressure on the bordering tissues, solidly fixing the tool in the knee.

Balloons can be inflated by gas or liquid. For two main reasons the use of physiological liquid is preferred:

The first one is a safety reason. Physiological liquid, a mixture of water and different salts, is widely used in medicine. If the balloons of the inventive device with a medical tool such as e.g. a nanoinstrument are activated with the same liquid, no harm is expected in case of a leak.

The second reason is that vibrations are more effectively damped in a liquid. Therefore using physiological solution to inflate balloons has the additional advantage of absorbing small movements and vibrations around of the tool. The feeding of the liquid and/or gas through the capillary tubes 5 to inflate the balloons 3,4 can e.g. be done by a pump.

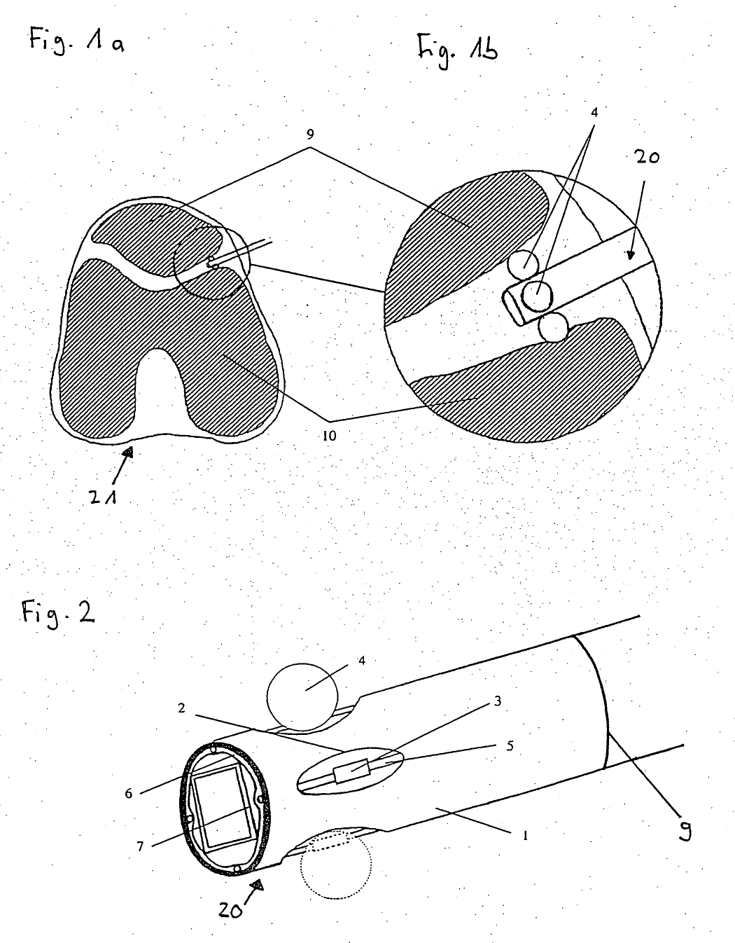

FIG. 2 shows a perspective view of an exemplary embodiment of the device 20 of the present invention.

In said embodiment the inventive device 20 comprises an elongated tubular structure 1 with oval surface openings 2, inflatable balloons 3,4 connected to capillary tubes 5 and located in the surface openings 2, a means 6 for forcing the balloons to expand outside the elongated tubular structure 1, a means 7 for receiving a medical tool and a connecting means 9.

The means 7 for receiving a medical tool can be any structure allowing a secure fixation of the medical tool to the elongated tubular structure 1. In a preferred embodiment said structure is a recess with a polygonal profile.

The capillary tubes 5 connected to the inflatable balloons 3,4 can be fixed to the inside of the elongated tubular structure 1 in such away that the balloons 3,4 are forced to expand outside the surface openings 2 of the tubular elongated structure 1.

The size, shape, number and arrangement of the surface openings 2 on the elongated tubular structure 1 can vary. The optimal arrangement of the surface openings 2 for a specific body cavity depends on the shape and size of the body cavity. To achieve a good stabilisation and/or positioning of a medical tool in a cavity with an uneven shape, the device 20 comprises preferably at least four surface openings 2. The centres of said surface openings 2 can e.g. be uniformly distributed along a circumference of the elongated tubular structure 1. When more than four surface openings 2 are present on the tubular structure 1, said surface openings can e.g. be arranged in two planes and be uniformly distributed along a circumference of the elongated tubular structure 1.

The size and shape of the surface openings 2 has to be large enough so that the inflated balloons 4 can pass through said openings 2 and get located on the surface of the elongated tubular structure 1. It is important that the surface openings 2 are arranged on the elongated tubular structure 1 in such a manner that they are inside the body cavity when the device 20 is inserted in the body cavity. In an exemplary embodiment of the inventive device the balloons 3,4 can be located outside the elongated tubular structure 1 and be connected to the capillary tubes 5 through the wall of the elongated tubular structure 1.

The elongated tubular structure 1 can comprise a first part which is for insertion in the body cavity and a second part which is for handling the device 20 outside the body wherein said two parts are connected by a connecting means 9. Such a connecting means 9 can e.g. be a bayonet joint or a thread. Said connecting means 9 allows the removal of the part of the elongated tubular structure 1 which is outside the body after the stabilisation and/or positioning of the medical tool in the body cavity has been achieved and said removal can further improve the stabilisation and/or positioning of the medical tool in the body cavity.

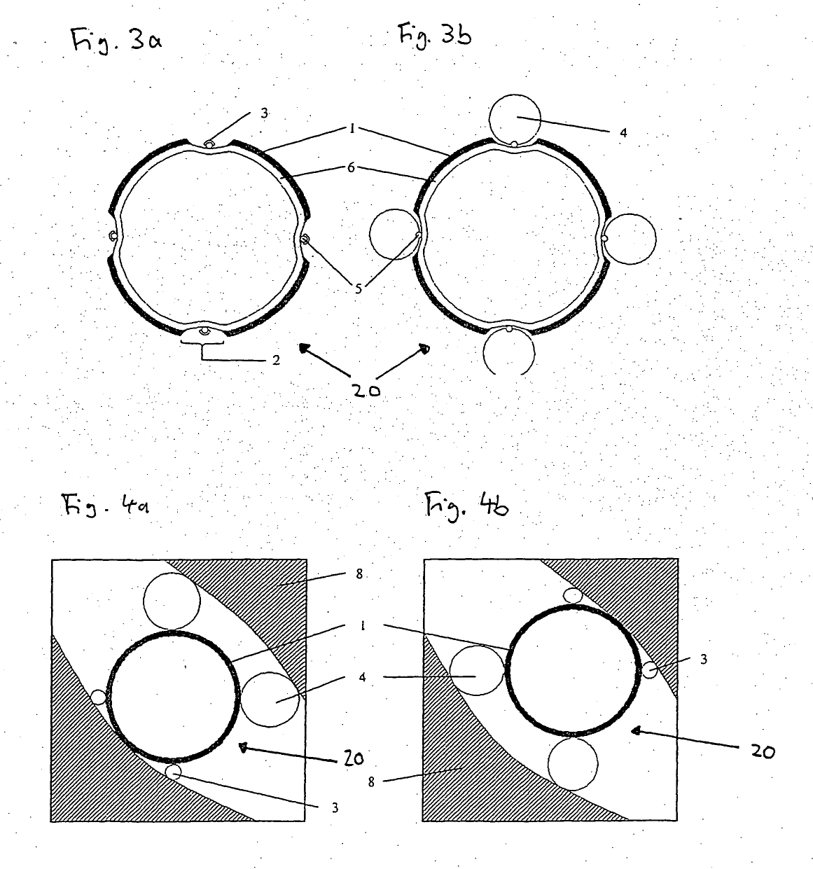

FIG. 3a shows a cross-sectional view of a device 20, with surface openings 2 and deflated balloons 3 connected to capillary tubes 5. In the shown embodiment the means 6 for forcing the inflated balloons outside the elongated tubular structure 1 is a ring which runs along the inner circumference of the elongated tubular structure 1. The person skilled in the art knows other suitable embodiments of structures for forcing the inflated balloons 4 outside the elongated tubular structure 1.

FIG. 3b shows a cross-sectional view of a device 20, with inflated balloons 4. The balloons 3,4 can comprise on their surface protuberances to achieve a good stabilisation on the inner surface of the body cavity or tissue.

FIG. 4a shows a cross-sectional view of a device 20 applied in an exemplary body cavity. The exemplary embodiment comprises four balloons 3,4 which have been inflated using different pressure.

FIG. 4b shows a cross-sectional view of a device 20 applied in a further exemplary body cavity. The exemplary embodiment comprises four balloons 3,4 which have been inflated using different pressure.

The embodiments of FIG. 4a and 4b show that the device 20 of the present invention allows for a positioning of medical tools in body cavities with variable shapes. Depending on the shape of the cavity different pressures can be used to inflate the single balloons 3,4 in order to achieve a stable position of the medical tool in the body cavity. The inflatable balloons 3,4 can comprise on their surface protrusions in order to optimise the attachment of the inflated balloons 4 to e.g. a rough surface of a bone.

Materials that can be used for the construction of the inventive device are known to a person skilled in the art. The elongated tubular structure can e.g. be made of any biocompatible material such as e.g. stainless steel. The capillary tubes 5 can e.g. be made of the material used to produce heart catheters.

While there are shown and described presently preferred embodiments of the invention, it is to be distinctly understood that the invention is not limited thereto but may be otherwise variously embodied and practised within the scope of the following claims.

1. A medical instrument comprising:
an elongated tubular structure with an end for insertion in a body cavity;
at least one inflatable balloon connected to at least one capillary tube and inflatable by pressing a fluid and/or gas into said tube.
an atomic force microscope; and
a means for receiving said atomic force microscope.

2. The instrument of claim 1, wherein said at least one balloon is arranged at a distance from said atomic force microscope.

3. The instrument of claim 1, wherein said elongated tubular structure further comprises at least one surface opening and said at least one inflatable balloon is located at said at least one surface opening.

4. The instrument of claim 3, further comprising several surface openings, wherein said surface openings are uniformly distributed along a circumference of said elongated tubular structure and the centres of all surface openings are at equal distance from the end of the elongated tubular structure to be inserted in the body cavity.

5. The instrument of claim 1, wherein said means for receiving said atomic force microscope is a recess having a polygonal profile.

6. The instrument of claim 1, wherein said elongated tubular structure further comprises a means for forcing the balloons to expand outside said elongated tubular structure.

7. The instrument of claim 6, wherein said means for forcing the balloons to expand outside said elongated tubular structure is a ring.

8. The instrument of claim 6, wherein said capillary tubes are fixed to said means for forcing the balloons to expand outside said elongated tubular structure.

9. (canceled)

10. The instrument of claim 1, wherein said elongated tubular structure comprises a first part and a second part which are connected by a connecting means.

11. (canceled)

12. A method for stabilising and/or positioning an atomic force microscope in a body cavity comprising the steps of:
[I]introducing a device comprising said atomic force microscope and at least one inflatable balloon arranged in a distance from said atomic force microscope in a body cavity,
inflating said at least one balloon with a liquid and/or gas until said at least one inflated balloon contacts an inner surface of the body cavity and said atomic force microscope is stabilised and/or positioned.

13. The method of claim 12, wherein the at least one inflatable balloon is inflated by a liquid, preferably a physiological liquid.

14. The method of claim 12, wherein said device comprises at least two balloons, preferably at least four balloons.

15. The method of claim 12, wherein said body cavity is a joint.

16. The method of claim 15, wherein the joint is the knee joint.

17. The instrument of claim 3, wherein said elongated tubular structure comprises at least two surface openings and at least two inflatable balloons located at said surface openings.

18. The instrument of claim 3, wherein said elongated tubular structure comprises at least four surface openings and at least four inflatable balloons located at said surface openings.

19. The instrument of claim 1 comprising several balloons, wherein said balloons are inflatable by different pressures.

20. The method of claim 14 wherein said device comprises at least four balloons.

21. A medical instrument comprising:
an elongated tubular structure with an end for insertion in a body cavity;
at least one inflatable balloon connected to at least one capillary tube and inflatable by pressing a fluid and/or gas into said tube, and
an atomic force microscope at said end of said tubular structure.
